(19) United States
(12) Patent Application Publication
(10) Pub. No.: US 2008/0090299 A1
(43) Pub. Date: Apr. 17, 2008

(54) Method and system for identification of protein-protein interactions

(76) Inventor: James Alexander Apffel, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/580,857

(22) Filed: Oct. 16, 2006

(51) Int. Cl. G01N 33/00

(52) U.S. Cl. 436/86; 436/173

(57) ABSTRACT

A method for rapid detection and possibly identification of protein complexes is disclosed. The method utilizes a two stage high resolution chromatographic analysis and a reversible crosslinker to detect and identify protein complexes. The identification of protein complexes may be further improved by mass spectrometry analysis of chromatographic fractions containing the complexes. A system for implementing the method is also provided.

### TECHNICAL FIELD

The invention relates generally to protein analysis method and more particularly to rapid and high resolution detection and identification of protein complexes using diagonal chromatography.

### BACKGROUND OF THE INVENTION

Protein-protein interactions constitute an important part of the molecular mechanism of biological processes. One method for detecting protein-protein interactions is diagonal gel electrophoresis (see e.g., Brennan et al., J Biol Chem 2004, 279:41352-41360). In this technique, interacting proteins are cross-linked in vivo or in vitro, usually using disulfide formation between cysteines. The mixture, containing crosslinked complexes is then separated by size with a first dimension sodium dodecyl sulfate polyacrylamide gel electrophoresis (SDS-PAGE). The disulfide bonds are then reduced and the mixture is re-separated by size with SDS-PAGE. In the second dimension of separation, all components that were originally single proteins, unassociated with any complex, migrate the same as in the first dimension, forming a diagonal pattern in the two-dimension (2D) separation. The components of the complexes that were originally bound together are now unbound and will migrate independently, off the diagonal. Conceptually, this approach sounds relatively simple and elegant. However, it suffers from a number of specific drawbacks that have resulted in low adoption rate. In practice, the use of gel electrophoresis has been limited in terms of resolution and the information produced directly from the electrophoresis experiment has been insufficient to identify the interacting proteins, require additional analytical steps for identification. Furthermore, limitations inherent to gel electrophoresis such as sample solubility, speed and automation issues still hamper the usefulness of this approach.

A chromatographic implementation of diagonal separations has been used for purifying proteins for proteomics (see e.g., Gevaert et al., Mol Cell Proteomics 2002, 1:896-903; Gevaert et al., Nature Biotechnology 2003, 21:566-569; Gevaert et al., Proteomics 2004, 4:897-908; Staes et al., Journal of Proteome Research 2004, 3:786-791; Gevaert et al., Anal Biochem 2005, 345:18-29; Gevaert et al., Proteomics 2005, 5:3589-3599; Martens et al., Proteomics 2005, 5:3193-3204; Van Damme et al., Nat Methods 2005, 2:771-777). This approach is based on initially separating a mixture of proteins or peptides by a first dimension and then causing a chemical modification to those proteins or peptides and then separating them by a second dimension. Proteins or peptides subject to modification can be identified by the fact that they elute of the diagonal of the 2D separation space. This technique, however, has not been applied to protein-protein interactions.

Other approaches for characterization protein-protein interactions, such as Yeast-Two Hybrid, Tandem Affinity Probe-Mass Spectrometry and many in vivo tagging procedures, require costly or time consuming experimental preparations, such as the preparation of specific antibodies, genetic constructs or protein translation systems to characterize interactions of specific target-bait interactions.

Therefore, the need remains for an assay method that can rapidly detect and identify protein complexes with high resolution.

### SUMMARY OF THE INVENTION

One aspect of the present invention relates to a method for identifying protein-protein interactions. The method comprises: crosslinking interacting proteins, subjecting crosslinked proteins to a first dimension chromatographic analysis and collecting fractions; un-crosslinking proteins in the collected fractions, subjecting the un-crosslinked proteins to a second dimension chromatographic analysis under conditions substantially identical to that of the first dimension chromatographic analysis, and constructing a two dimension chromatogram by plotting data of the second dimension chromatographic analysis against data of the first dimension chromatographic analysis.

In one embodiment, the method further comprises the step of identifying proteins in off-diagonal fractions by mass spectrometry analysis.

In another embodiment, the method further comprises the step of integrating data from the first and the second chromatographic analyses with data from the mass spectrometry to reconstitute a protein complex.

In another embodiment, the method further comprises the step of isolating and concentrating a sub-proteomic fraction of crosslinked proteins prior to the first dimension chromatography.

In another embodiment, the method further comprises the step of removing a reducing agent from the un-crosslinked proteins prior to the second dimension chromatographic analysis.

In another embodiment, the first dimension chromatographic analysis and the second dimension chromatographic analysis are coupled with electrospray ionization time-of-flight mass spectrometry (ESI-TOF MS) analysis for determination of molecular weight in parallel to the fraction collection process.

In yet another embodiment, the first chromatography analysis and the second chromatographic analysis are performed using macroporous reversed phase HPLC columns.

Another aspect of the present invention relates to a system for identifying protein-protein interactions. The system comprises: a chromatographic unit capable of high resolution separation of protein molecules, a mass spectrometry (MS) unit coupled to the chromatographic unit for identifying proteins in chromatographic fractions, and a data acquisition system capable of collecting a first set of chromatographic data, a second set of chromatographic data, and a set of MS data, plotting the first set of chromatographic data versus the second set of chromatographic data to detect components of a protein complex, and integrating the chromatographic data with the MS data to identify components of the protein complex.

In one embodiment, the MS unit is a LC-MS/MS unit.

In another embodiment, the system further comprises a second MS unit coupled to the chromatographic unit for determining molecular weights of proteins in chromatographic fractions.

### DETAILED DESCRIPTION OF DRAWINGS

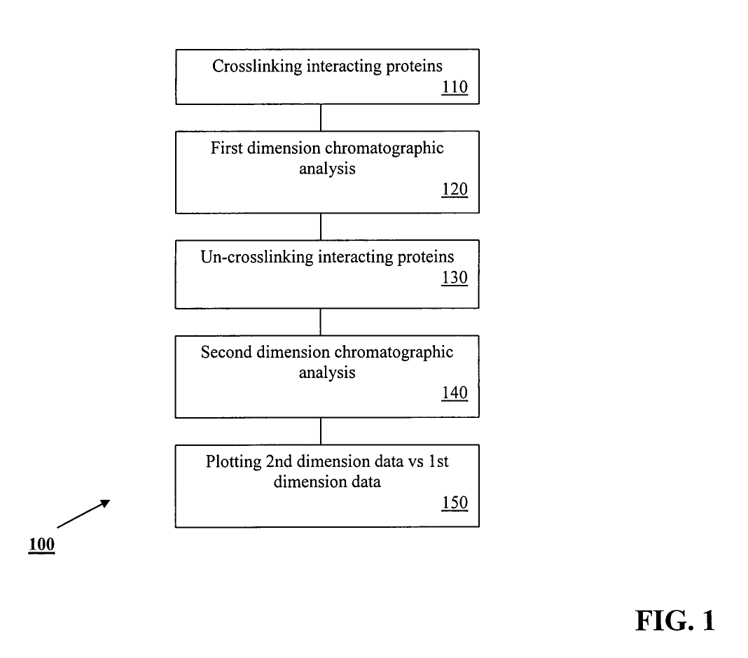

FIG. 1 is a block diagram showing an embodiment of the diagonal chromatographic method for identification of protein-protein interactions.

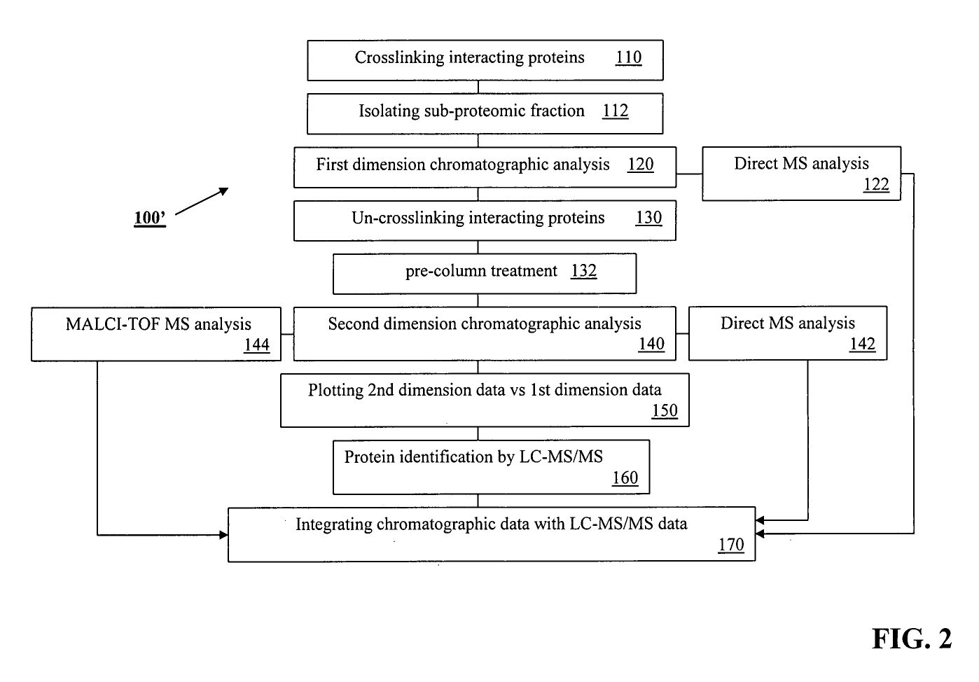

FIG. 2 is a block diagram showing another embodiment of the diagonal chromatographic method for identification of protein-protein interactions.

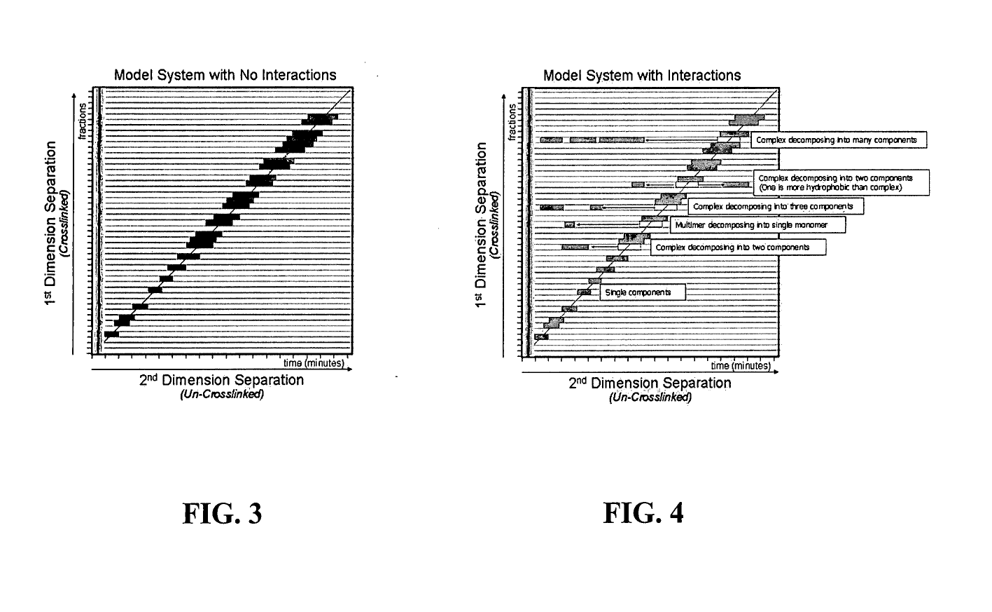

FIG. 3 is a schematic showing a hypothetic result of diagonal chromatography of proteins with no interactions.

FIG. 4 is a schematic showing a hypothetic result of diagonal chromatography of proteins with interactions.

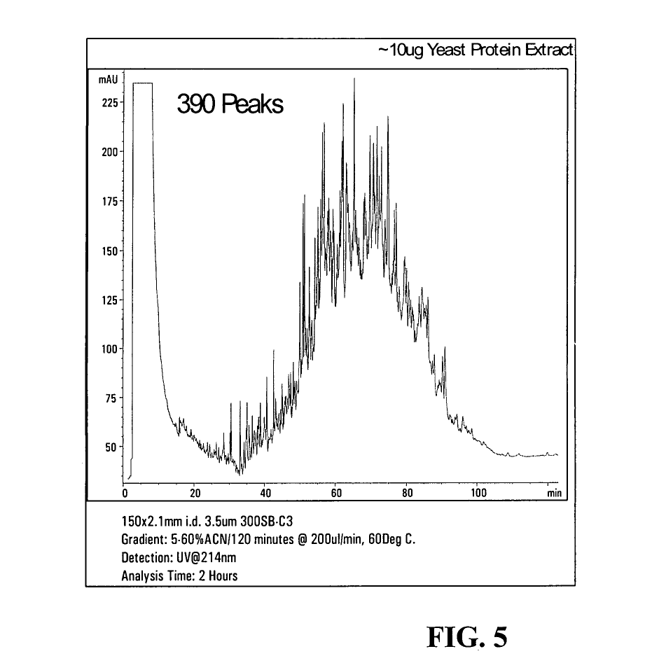

FIG. 5 is a representative chromatogram showing the resolution of reversed phase chromatography.

### DETAILED DESCRIPTION OF THE INVENTION

The present invention utilizes a two stage chromatographic analysis and a reversible crosslinker for rapid detection and identification of protein complexes. The detected protein complexes can be further identified by mass spectrometry analysis.

FIG. 1 shows an embodiment of the diagonal chromatographic method 100 of the present invention. The method 100 contains the steps of crosslinking interacting proteins (110), subjecting a protein sample from step 110 to a first dimension chromatographic analysis and collecting fractions (120), un-crosslinking the collected fractions (130), subjecting the un-crosslinked fractions to a second dimension chromatographic analysis (140) under conditions substantially identical to that of the first dimension chromatographic analysis, and constructing a 2D chromatogram by plotting the second dimension data against the first dimension data (150).

The crosslinking step 110 may be performed in vitro or in vivo. In one embodiment, the crosslinking is performed in vitro. This procedure involves the formation of covalent bonds between two proteins by using bifunctional reagents containing reactive end groups that react with functional groups, such as primary amines and sulfhydryls, of amino acid residues. If two proteins interact with each other, they can be covalently crosslinked. The formation of crosslinks between two distinct proteins is a direct evidence of their close proximity.

A wide range of crosslinking reagents are commercially available from major suppliers such as Pierce (Rockford, Ill.), Molecular Probes (Eugene, Oreg.), and Sigma (St. Louis, Mo.). The crosslinking reagents can be either homo- or hetero-bifunctional reagent with identical or non-identical reactive groups, respectively. Examples of homo-bifunctional crosslinking reagents include, but are not limited to, glutaraldehyde, imidoesters such as dimethyl adipimidate (DMA), dimethyl suberimidate (DMS), and dimethyl pimelimidate (DMP) with spacer arms of various lengths between the reactive end groups.

In one embodiment, the crosslinking reagent is a reversible home-bifunctional crosslinkers. Examples of reversible home-bifunctional crosslinkers include, but are not limited to, N-hydroxysuccinimide (NHS) esters such as dithiobis(succinimidylpropionate) (DSP) and dithiobis(sulfosuccinimidylpropionate) (DTSSP), and Bis[2-(Succinimidooxycarbonyloxy)ethyl]sulphone (BSOCOES). These crosslinkers can be cleaved by treatment with thiols, such as β-mercaptoethanol or dithiothreitol.

In contrast to homo-bifunctional crosslinking reagents, hetero-bifunctional crosslinkers have two different reactive groups. In one embodiment, the crosslinking reagent is a hetero-bifunctional crosslinker having one amine-reactive end and a sulfhyfryl-reactive moiety. In another embodiment, the crosslinking reagent is a hetero-bifunctional crosslinker having a NHS ester at one end and an SH-reactive group, such as maleimide or pyridyl disculfide, at the other end. In another embodiment, the crosslinking reagent is a hetero-bifunctional crosslinker having a photoreactive group, such as Bis[2-(4-azidosalicylamido)ethyl] disulfide (BASED).

In another embodiment, the crosslinking reagent is sulfo-SFAD (Sulfosuccinimidyl-[perfluoroazidobenzamido]ethyl-1,3′-dithiopropionate) (Pierce Chemical, Rockford, Ill.). Sulfo-SFAD is a heterobifunctional crosslinking reagent. Exposed amine groups in proteins can be reacted with the NHS-Ester moiety of the reagent. The crosslinking can also be initiated through photoconjugation by radiation at 320 nm for reaction with a halogen substituted phenylazide group at the other end. The two reactive groups are joined by a cleavable disulfide linkage, so the crosslinking can be reversed by reduction. The reagent is water soluble, couples with high efficiency and has a spacer arm of approximately 15 Å in length.

In another embodiment, the crosslinking reagent is a heterotrifunctional crosslinking reagent having two reactive groups that can be used to crosslink interacting proteins and a third reactive group (e.g., biotin) that can be used as selective isolation group (e.g., for streptavidin pull-down). In this embodiment, the affinity portion of the crosslinking reagent is used to selectively isolate only those proteins that were involved in chemical crosslinking reactions. Non-interacting proteins would be washed away and would not be subjected to the first dimension separation.

In another embodiment, the crosslinking step 110 is performed in vivo. In vivo crosslinking offers the advantage of capturing both stable and transient interactions in a biologically relevant context with a minimal perturbation to the system under study. In vivo crosslinking would effectively take a snapshot of the system at a given point in time. However, in vivo crosslinking requires that the crosslinking reagent be cell permeable, the crosslinking can be initiated, and the crosslinking reaction be reversible. Examples of in vivo crosslinking reagents include, but are not limited to, formaldehyde and BSOCOES.

A sample of crosslinked proteins is then prepared for the first dimensional chromatographic analysis. The crosslinked sample typically contains a mixture of individual proteins and crosslinked protein complexes. As shown in FIG. 2, an optional pre-column treatment step 112 may be added to isolate and concentrate the proteins of interest. For example, if the proteins of interest are known to be located on the endoplasmic reticulum (ER), the sample can be enriched for the ER fraction by density gradient separation.

The first dimension chromatography is carried out using high performance liquid chromatography (HPLC), fast protein liquid chromatography (FPLC) or other comparable chromatographic techniques. In one embodiment, the first dimension chromatography is performed using macroporous reversed phase (mRP) HPLC columns because their high resolution, high recovery and potentially high speed. Chromatographic conditions, such as column, stationary phase, mobile phases, gradient, temperature, flow rate, etc. are determined based on the sample content and the characteristics of the proteins of interest. One skilled in the art would recognize that a range of chromatographic modes can be used in method 100.

The chromatographic conditions should be selected in favor of the highest resolution because high resolution separations directly effect the peak capacity. Furthermore, for a given sample complexity, the resolution is directly related to the speed of the separation. The total protocol analysis time may be expressed by the following formula:

T₀=T₁+nT₂

where T₀ is the total analysis time, T₁ is the time of the first dimension analysis, T₂ is the time of the second dimension analysis, and n is the number of first dimension fractions. Accordingly, in order to increase throughput, fast chromatographic methods should be used and fewer first dimension fractions may be collected (i.e., limiting digitization of the first dimension).

If a large volume is collected as a fraction late in a reversed phase run in the 1^{st} dimension, injecting that fraction into the 2^{nd} dimension will result in injection of a large volume of organic solvent at the beginning of a gradient. The high organic content may cause poor resolution and a lack of peak focus. In one embodiment, this problem is overcome by either evaporating the organic from the collected fractions using a vacuumed centrifuge system, such as the SpeedVac system from Thermo Electron Corp. (Waltham, Mass.), following reversal of the crosslinking reaction or by diluting the organic with water to <10% weight/weight. The diluted fractions can be injected onto a reversed phase (or other retentive stationary phase) column, which would allow large volumes of sample (or collected fractions) to be injected into the column without any band broadening effect. Use of a retentive stationary phase, however, is not strictly necessary.

During the chromatographic process, fractions of the effluent are collected on a fixed volume or fixed time basis. In one embodiment, the first dimension chromatography is directly coupled, in parallel via a flow splitter, to ESI-TOF MS for direct measurement of molecular weights of proteins and protein complexes (FIG. 2, step 122).

The un-crosslinking of the collected fractions (step 130) can be accomplished by adding a reducing reagent to reverse the crosslinking of proteins. The reducing reagent is selected based on the crosslinking reagent used in the crosslink step 110. For example, if sulfo-SFAD (Sulfosuccinimidyl-[perfluoroazidobenzamido]ethyl-1,3′-dithiopropionate) is used as the crosslinking reagent. The reducing reagent can be β-mercaptoethanol or dithiothreitol. The un-crosslinking of the collected fractions may also be achieved with a photo or thermo process. In the case of BSOCOES, the removal of the crosslink is performed by raising the pH to >8.5. For example, the use of formaldehyde for a non-specific crosslinking can be reversed by heating the samples to temperature above 68° C. Similarly, photocleavable crosslinkers can reverse the complex crosslinking upon irradiation. For example, the use of photocleavable biotin has be used in DNA synthesis applications.

The second dimension chromatography is performed under conditions substantially identical to that of the first dimension chromatography. As used herein, the term “substantially identical conditions” refers to chromatographic conditions that are maintained as identical to each other as possible in a particular experimental setting. For example, the first and the second dimension chromatograpic analysis should be performed with the same instrument (i.e., the same machine, column, detector, etc.) and the same settings (i.e., the same mobile phase, gradient, temperature, flow rate, fraction volume, etc.). As shown in FIG. 2, a pre-column treatment step 132 may be performed. In one embodiment, the high organic content in the first dimension fractions is removed or diluted prior to the second dimensional chromatographic analysis. In another embodiment, the reducing reagent in the un-crosslinked fractions is removed prior to the second dimensional chromatographic analysis using, for example, a spin column.

Effluent fractions from the second chromatography may be collected on a fixed time or volume basis. In one embodiment, a small portion of the second dimension chromatographic effluent is be split and introduced directly into an ESI-TOF MS instrument to generate molecular weight information on the interacting components (FIG. 2, step 142). For large entities with multiple charge states, the molecular weight information may be obtained following spectral charge-state deconvolution. In another embodiment, small volumes of the second dimension chromatographic effluent are spotted onto plates and analyzed by matrix-assisted laser desorption/ionization time-of-flight mass spectroscopy (MALDI-TOF MS) for molecular weight determination. (FIG. 2, step 144).

In step 150, data from the second dimension chromatographic analysis is ploted against the data from the first dimension chromatographic analysis in the order of the fraction collection. In one embodiment, the data is extracted as csv (comma separated variable) files and processed through a specific visualization software to effectively plot abundance at a specific wavelength as a function of the retention by both dimensions. As shown in FIG. 3, proteins that are not associated with complexes would elute with identical retention times for both dimensions and appear along a diagonal. Proteins that were members of a complex would be crosslinked and eluted as a single peak in the first dimension, but following un-crosslinking, they will appear as multiple peaks in the second dimension. These proteins will thus appear off diagonal in the plot (FIG. 4).

For those self-aggregating proteins that form multimers, they would appear as single peaks in both the first dimension and the second dimension (assuming complete un-crosslinking.) However, they will still elute off-diagonal because they would have different retention times in the first dimension analysis (as multimers with higher molecular weights) and the second dimension analysis (as monomers with lower molecular weights).

In one embodiment, size exclusion chromatography (SEC) is used in steps 120 and 140. The retention time may be correlate with molecular weight. In another embodiment, macroporous reversed phase chromatography, which provides a better resolution and higher speed than SEC, is used as the separation principle. In this case, dissociated proteins may be eluted earlier or later than the complex to which they originally belong, since their retention time will depend on their hydrophobicity rather than their molecular weight. To the extent that there is a correlation of hydrophobicity to molecular weight, the components may tend to elute before the interact complex.

In principle, the digitization of the second dimension separation can be very high (1-5 Hz), but the digitization of the first dimension is limited for practical considerations. Specifically, the more fractions that are collected, the finer the resolution of the first dimension separation, but the more second dimension separation will have to be run and the longer the whole experiment will take. Furthermore, the fraction collector's capacity (typically 4×96 well plates) and its movement speed may limit the number of fractions collected in each dimension.

To identify the interacting proteins in a complex, off-diagonal fractions are reduced, alkylated, digested and subjected to mass spectrometry analysis (FIG. 2, step 160). In one embodiment, step 160 is performed with LC-MS/MS. The MS data can be integrated with 2D plot data to positively idenfity the components of protein complexes (FIG. 2, step 170).

The method of the present invention will have an optimal window of applicability in terms of mixture complexity. This will depend on the resolution of the chromatographic separation used. This, in turn, has ramifications in terms of separation speed and total analysis time. One of the advantages of multidimensional separations is the enhanced separation peak capacity. In the first dimension, protein complexes are being separated. In the second dimension, individual proteins are being separated. Thus, the overall separation peak capacity is composed of two separate peak capacities: the maximum number of protein complexes that might be separated by the first dimension (n₁) and the maximum number of components that can be separated for any single complex by the second dimension (n₂).

Peak capacities depend on the chromatographic modes and conditions utilized. In one embodiment, the chromatographic analysis of the present invention could resolve 100-150 proteins in 30-60 minutes using a reversed phase chromatographic material. As shown in FIG. 5, a reversed phase chromatographic material is capable of resolving nearly 400 peaks in 90 minutes (FIG. 5). In another embodiment, the chromatographic system has a large sample capacity and a high recovery rate for detection of interacting proteins with low abundance.

The method of the present invention may be implemented in a miniaturized or microfluidic format, in order to minimize the quantity of samples required for the protein complex analysis. In one embodiment, the detection system uses an UV/VIS detector and is capable of performing an analysis with 1-10 ng of protein. In another embodiment, the detection system uses mass spectrometry as an on-line detector and is capable of performing an analysis with proteins in the range of sub-femto moles. The detection scale and capacity can be adjusted for each application such that enough original material can be introduced and separated by the system to detect components of interest.

In an embodiment, a portion of the effluent from both the first and second dimension is sampled directly by ESI-TOF MS. For a typical analysis, conventional (4.6 mm i.d.) or narrow (2.1 mm i.d.) bore mRP columns are used. The vast majority (99%+) of the effluent can be collected with a fraction collector while a very small proportion at 1-5 μL/min can be introduced via nanospray or ChipMS infusion chip directly into the ESI-TOF MS. Following computational deconvolution of the multiple charged ion spectra produced by proteins in ESI, the data obtained from the first dimension would give an indication of the molecular weight of the intact protein complexes, while data from the second dimension would give the molecular weights of the individual components of complex. All components in a given fraction can be associated with the same complex.

The molecular weight data generated by the mass spectrometry analysis can be used in conjunction with the peptide-level bottom up data obtained following digestion and LC-MS/MS to add confidence to protein identification (FIG. 2, step 170). Comparing the two sets of data yields additional information; the molecular weight data information on the actual protein (including post-transcription modifications (PTM's)), whereas the peptide based IDs show molecular weights based on amino acid sequences alone. Thus inferences can be made about the character and nature of the PTMs based on the difference. These inferences can be further investigated from the raw LC-MS/MS data directly.

Intact protein molecular weight data usually is not specific enough to identify a protein unequivocally. In one embodiment, a Linear Ion Trap-Time-of-Flight (LT-TOF) Mass Spectrometer is used to obtain MS-based top-down sequencing data. The LT-TOF MS is interfaced directly to the chromatographic instrument for the first and second dimension analysis. In this approach, intact proteins are fragmented directly in the gas phase in the mass spectrometer. The structurally significant fragments are used to identify the protein.

In coupling the MS to the chromatographic analysis, a balance must be made between chromatographic performance and MS compatibility. In the case of a reversed phase separation, mixtures of water and acetonitrile present no problem. However, the added acid can be problematic. Best chromatographic resolution for proteins is typically obtained with approximately 0.1% trifluoroacetic acid (TFA), but this is know to suppress ESI ionization efficiency. Formic acid may be used to replace TFA, but formic acid may introduce reduced chromatographic resolution and performance. In one embodiment, the mobile phase is composed of 0.1% Formic Acid and 0.01% TFA. A post-column, post-UV detector, pre-fraction collector split can be easily achieved with low dead volume splitters that are commercially available.

Another embodiment provides a system for identifying protein-protein interactions. The system comprises a chromatographic unit capable of high resolution separation of protein molecules, a mass spectrometry (MS) unit coupled to the chromatographic unit for identifying proteins in chromatographic fractions, and a data acquisition system capable of collecting a first set of chromatographic data, a second set of chromatographic data, and a set of MS data. The data acquisition system plots the first set of chromatographic data versus the second set of chromatographic data to detect components of a protein complex and integrates the chromatographic data with the MS data to identify components of the protein complex.

In one embodiment, the chromatographic unit is a reverse phase chromatographic unit and the MS unit is a LC-MS/MS unit.

In another embodiment, the system further comprises a second MS unit coupled to the chromatographic unit for determining molecular weights of proteins in chromatographic fractions.

The foregoing discussion discloses and describes many exemplary methods and embodiments of the present invention. As will be understood by those familiar with the art, the invention may be embodied in other specific forms without departing from the spirit or essential characteristics thereof. Accordingly, the disclosure of the present invention is intended to be illustrative, but not limiting, of the scope of the invention, which is set forth in the following claims.

1. A method for identifying protein-protein interactions, comprising:
crosslinking interacting proteins;
subjecting the crosslinked proteins to a first dimension chromatographic analysis and collecting fractions;
un-crosslinking proteins in the collected fractions;
subjecting the un-crosslinked proteins to a second dimension chromatographic analysis under conditions substantially identical to that of the first dimension chromatographic analysis; and
constructing a two-dimension chromatogram by plotting data of the second dimension chromatographic analysis against data of the first dimension chromatographic analysis.

2. The method of claim 1, further comprising the step of identifying proteins in off-diagonal fractions of the two-dimension chromatogram by mass spectrometry analysis.

3. The method of claim 2, wherein the mass spectrometry analysis is LC-MS/MS analysis.

4. The method of claim 2, further comprising integrating data from the first dimension and the second dimension chromatographic analyses with data from the mass spectrometry to reconstitute a protein complex.

5. The method of claim 1, further comprising isolating and concentrating a sub-proteomic fraction of crosslinked proteins prior to subjecting the crosslinked proteins to the first dimension chromatographic analysis.

6. The method of claim 1, wherein subjecting the crosslinked proteins to the first dimension chromatographic analysis includes coupling the first dimension chromatographic analysis with ESI-TOF MS analysis for determination of molecular weight.

7. The method of claim 1, further comprising removing a reducing agent from the un-crosslinked first dimension chromatographic fractions prior to the second dimension chromatographic analysis.

8. The method of claim 1, further comprising removing organic solvent from the un-crosslinked first dimension chromatographic fractions prior to subjecting the un-crosslinked proteins to the second dimension chromatographic analysis.

9. The method of claim 1, wherein subjecting the un-crosslinked proteins to the second dimension chromatographic analysis includes coupling the second dimension chromatographic analysis with ESI-TOF MS analysis or MALDI-TOF MS analysis for determination of molecular weight.

10. The method of claim 1, wherein the crosslinking is performed in vitro.

11. The method of claim 10, wherein the crosslinking is performed using sulfo-SFAD.

12. The method of claim 1, wherein the crosslinking is performed using a heterotrifunctional reagent, and wherein the crosslinked proteins are isolated prior to subjecting the crosslinked proteins to the first dimension chromatographic analysis.

13. The method of claim 1, wherein the crosslinking is performed in vivo.

14. The method of claim 13, wherein the crosslinking is performed using Bis[2-(Succinimidooxycarbonyloxy)ethyl]sulphone (BSOCOES).

15. The method of claim 1, wherein the first chromatographic analysis and the second chromatographic analysis are performed using macroporous reversed phase HPLC columns.

16. The method of claim 1, wherein the first chromatographic analysis and the second chromatographic analysis are performed using size exclusion HPLC columns.

17. A method for identifying protein-protein interactions, comprising:
crosslinking interacting proteins;
isolating a sub-proteomic fraction of crosslinked proteins;
subjecting the isolated, crosslinked proteins to a first dimension chromatographic analysis and collecting fractions;
un-crosslinking proteins in the collected fractions;
removing undesired reagents from the un-crosslinked proteins;
subjecting the un-crosslinked proteins to a second dimension chromatographic analysis under conditions substantially identical to that of the first dimension chromatographic analysis;
constructing a two-dimension chromatogram by plotting data of the second dimension chromatographic analysis against data of the first dimension chromatographic analysis;
identifying proteins in off-diagonal fractions of the two-dimension chromatogram by mass spectrometry analysis; and
integrating data from the first and the second chromatographic analyses with data from the mass spectrometry to reconstitute a protein complex.

18. A system for identifying protein-protein interactions, comprising:
a chromatographic unit capable of high resolution separation of protein molecules;
a mass spectrometry (MS) unit coupled to the chromatographic unit for identifying proteins in chromatographic fractions;
a data acquisition system capable of collecting a first set of chromatographic data, a second set of chromatographic data, and a set of MS data, and capable of plotting the first set of chromatographic data versus the second set of chromatographic data to detect components of a protein complex and integrating the chromatographic data with the MS data to identify components of the protein complex.

19. The system of claim 18, wherein the chromatographic unit is a reverse phase chromatographic unit, and wherein the MS unit is a LC-MS/MS unit.

20. The system of claim 19, further comprising a second MS unit coupled to the chromatographic unit for determining molecular weights of proteins in chromatographic fractions.
